(19) United States
(12) Patent Application Publication
(10) Pub. No.: US 2024/0047071 A1
(43) Pub. Date: Feb. 8, 2024

(54) METHODS OF PREDICTING BONE FRACTURE RISK IN TYPE 2 DIABETES PATIENTS

(71) Applicant: Rensselaer Polytechnic Institute, Troy, NY (US)

(72) Inventors: Deepak Vashishth, Glenmont, NY (US); Bowen Wang, Albany, NY (US)

(73) Assignee: Rensselaer Polytechnic Institute, Troy, NY (US)

(21) Appl. No.: 18/216,679

(22) Filed: Jun. 30, 2023

Related U.S. Application Data
(60) Provisional application No. 63/357,185, filed on Jun. 30, 2022.

(51) Int. Cl. G16H 50/30; A61B 5/00; A61B 5/145

(52) U.S. Cl. CPC G16H 50/30; A61B 5/7275; A61B 5/4504; A61B 5/14532

(57) ABSTRACT

A method of predicting bone fracture risk in a type 2 diabetes (“T2D”) patient includes: obtaining a plurality of averaged glycated hemoglobin (“HbA1c”) values for a plurality of T2D subjects over an observational period; binning the plurality of averaged HbA1c values into two predetermined categories; obtaining a plurality of total fracture incidences for the plurality of T2D subjects over a follow-up period; performing a plurality of linear regression model correlations between the binned HbA1c values and the plurality of total fracture incidences to determine a bone fracture rate prediction model; obtaining a measurement of an HbA1c value of the T2D patient; and analyzing the HbA1c value of the T2D patient with the bone fracture rate prediction model to determine the T2D patient's risk of bone fracture for a future period.

### CROSS REFERENCE TO RELATED APPLICATIONS

This application claims the priority benefit of U.S. Provisional Patent Application No. 63/357,185, filed Jun. 30, 2022, which is incorporated by reference as if disclosed herein in its entirety.

### STATEMENT REGARDING FEDERALLY SPONSORED RESEARCH OR DEVELOPMENT

The present technology was developed with Government support under Grant No. R21 AR071681 awarded by the National Institutes of Health. The Government has certain rights in the technology.

### FIELD

The present echnology relates generally to the field of type 2 diabetes management and treatment, and more particularly, to methods of predicting bone fracture risk in type 2 diabetes patients.

### BACKGROUND

Despite elevated bone mineral density (“BMD”), people with type 2 diabetes (“T2D”) are at greater risk for fracture which is underestimated by current standard of care tools. The underlying pathogenesis of T2D fractures is complex and involves factors beyond BMD. For example, lack of glycemic control is correlated with various diabetic comorbidities in people with T2D, one of which is poor bone quality. The altered glucose metabolism disrupts bone turnover and negatively impacts bone material properties, which may lead to higher risk of fracture. Other T2D comorbidities, such as retinopathy, nephropathy, and hypertension, have also been shown to elevate the fracture risk. However, the duration and severity of T2D with its comorbidities are difficult to quantify. Glycated hemoglobin (“HbA1c”), as a reflection of glycemic control and an independent indicator for T2D status, has been widely used to assess the risk of diabetic comorbidities and mortality. The relationship between HbA1c and fracture risk is yet to be determined.

The measurement of HbA1c provides the average blood glucose level over the preceding 2 to 3 months. However, recent clinical studies have indicated that the deteriorated bone quality and elevated fracture risk may be related to high glycemic level over a prolonged period. Consequently, current methods evaluating the relationship between single time point measures of HbA1c (prior to fracture) with fracture incidences have not proven useful. Additionally, treatments targeting T2D and anti-osteoporosis medications may impact the risk of fracture. However, there is conflicting and insufficient evidence regarding assessing the impact of common treatments on fracture risk.

What is needed, therefore, is an improved method of predicting the fracture risk in T2D patients that addresses at least the problems described above.

### SUMMARY

According to an embodiment of the present technology, a method of predicting bone fracture risk in a type 2 diabetes (“T2D”) patient is provided. The method includes: obtaining a plurality of averaged glycated hemoglobin (“HbA1c”) values for a plurality of T2D subjects over an observational period; binning the plurality of averaged HbA1c values into two predetermined categories; obtaining a plurality of total fracture incidences for the plurality of T2D subjects over a follow-up period; performing a plurality of linear regression model correlations between the binned HbA1c values and the plurality of total fracture incidences to determine a bone fracture rate prediction model; obtaining a measurement of an HbA1c value of the T2D patient; and analyzing the HbA1c value of the T2D patient with the bone fracture rate prediction model to determine the T2D patient's risk of bone fracture for a future period.

In some embodiments, the two predetermined categories include a by every 1% change in HbA1c value category and a by binary change in HbA1c value category.

In some embodiments, the by every 1% change in HbA1c value category includes a 6% to 7% change bin, a 7% to 8% change bin, an 8% to 9% change bin, a 9% to 10% change bin, a 10% to 11% change bin, an 11% to 12% change bin, and a greater than or equal to 12% change bin.

In some embodiments, the by binary change in HbA1c value category includes an adequate glycemic control bin and a poor glycemic control bin.

In some embodiments, the adequate glycemic control bin includes an HbA1c value in the range of 6% to 9% and the poor glycemic control bin includes an HbA1c value greater than or equal to 9%.

In some embodiments, the plurality of linear regression model correlations includes a Kaplan-Meier survival estimation model and a Cox proportional hazards model. In some embodiments, the Kaplan-Meier survival estimation model is a univariate model and the Cox proportional hazards model is a multivariate model. In some embodiments, the multivariate model includes the variables of age, gender, comorbidities, glucocorticoids use, previous fragility fractures during the observational period, and prevalent T2D.

In some embodiments, the observational period is in the range of one year to three years and the follow-up period is in the range of one year to three years. In some embodiments, the observational period is two years and the follow-up period is two years.

In some embodiments, the future period is in the range of one year to three years. In some embodiments, the future period is two years.

In some embodiments, the method further includes: obtaining diabetic treatment medication information for the T2D patient; and adjusting the T2D patient's risk of bone fracture based on the diabetic treatment medication information.

In some embodiments, the diabetic treatment medication information includes metformin and the T2D patient's risk of bone fracture is decreased by about 12% from an initial value.

In some embodiments, the diabetic treatment medication information includes dipeptidyl peptidase-4 (“DDP4”) inhibitors and the T2D patient's risk of bone fracture is decreased by about 7% from an initial value.

In some embodiments, the diabetic treatment medication information includes meglitinides and the T2D patient's risk of bone fracture is increased by about 12% from an initial value.

In some embodiments, the diabetic treatment medication information includes thiazolidinediones (“TZDs”) and the T2D patient's risk of bone fracture is increased by about 20% to about 23% from an initial value.

In some embodiments, the diabetic treatment medication information includes insulin and the T2D patient's risk of bone fracture is increased by about 24% to about 26% from an initial value.

In some embodiments, the diabetic treatment medication information includes bisphosphonates and the T2D patient's risk of bone fracture is increased by about 15% from an initial value.

Further objects, aspects, features, and embodiments of the present technology will be apparent from the drawing Figures and below description.

### BRIEF DESCRIPTION OF DRAWINGS

Some embodiments of the present technology are illustrated as an example and are not limited by the figures of the accompanying drawings, in which like references may indicate similar elements.

FIG. 1 is a flowchart of a method for predicting bone fracture risk of a T2D patient according to some embodiments of the present technology.

FIG. 2 is a timeline chart of the population cohort data used in some embodiments of the present technology.

FIG. 3A shows the Kaplan-Meier estimation of unadjusted fracture risk with 95% CI stratified by 1% difference of HbA1c groups for 730 days of additional follow-up period.

FIG. 3B shows the “cross-section” of unadjusted fracture risk at the end of 2-year follow-up period, showing significant increasing linear correlation with the increase of longitudinal HbA1c percentage (P=0.003, R=0.93).

FIG. 3C shows the Kaplan-Meier estimation of unadjusted all fracture risk when longitudinal glycemic control is separated into 2 groups: adequate glycemic control (N=142,600) and poor glycemic control (N=14,839).

FIG. 3D shows the Kaplan-Meier estimation of unadjusted fragility fracture risk in poor and adequate glycemic control. The difference in fragility fracture risk between groups is not statistically significant (P=0.23).

FIG. 3E shows one year follow-up of fracture rate based on one-, two-, and three-year of HbA1c aggregation. As shown, two-year HbA1c predicts fracture risk in T2D better than one- and three-year HbA1c.

FIG. 4A shows the unadjusted fracture risk estimation by HbA1c mean in observational period, and HbA1c mean in follow-up period (up to first fracture incidence if applicable). HbA1c measured in the same period as fracture incidences underestimates the risk of all fractures. N=101,575 for mean HbA1c follow-up 6%-9%; N=11,266 for mean HbA1c follow-up 9%+.

FIG. 4B shows the unadjusted fracture risk estimation when stratified by high/low SD within each mean HbA1c level. The fracture risk is not statistically different when stratified by HbA1c SD. N=74,418 for mean HbA1c 6%-9%, SD<=0.5%; N=30,748 for mean HbA1c 6%-9%, SD>0.5%; N=1,968 for mean HbA1c 9%+, SD<=0.5%; N=7,522 for mean HbA1c 9%+, SD>0.5%.

FIG. 5A shows the unadjusted fracture risk estimation stratified by 4 racial groups (White population: N=99,056; Asian population: N=8,195; Black population: N=25,647, Hispanic population: N=14,888; Other/non-specified not considered).

FIG. 5B shows the White population as a reference point (hazard ratio set to 1) and the hazard ratios for other racial groups with covariates adjustment. Hazard ratio for Hispanic population: 0.93 (CI 0.91-0.95); for Black population: 0.81 (CI 0.79-0.84); for Asian population: (CI 0.55-0.68). All groups are significantly different from each other.

### DETAILED DESCRIPTION

Accordingly, exemplary embodiments of the present technology are directed to methods of predicting the bone fracture risk in type 2 diabetes (“T2D”) T2D patients. In some embodiments, the method includes determining the longitudinal relationship of glycated hemoglobin (“HbA1c”) with bone fractures in a large cohort of T2D patients for improving the risk assessment of T2D-induced fractures. In some embodiments, the method includes determining the associations of commonly used antidiabetic and anti-osteoporotic medications, with other risk factors adjusted, for managing fracture risk.

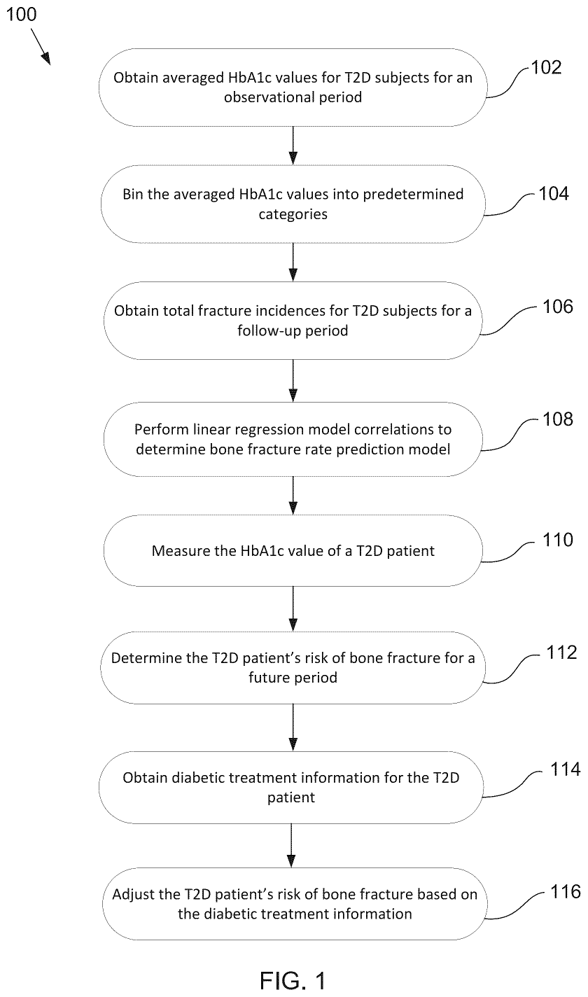

As shown in FIG. 1, a method of predicting the bone fracture risk in a T2D patient is generally designated by the numeral 100. At 102, the method 100 includes obtaining a plurality of averaged HbA1c values for a plurality of T2D subjects over an observational period. In some embodiments, the observational period is in the range of one year to three years. In some embodiments, the observational period is two years.

At 104, the method 100 includes binning the plurality of averaged HbA1c values into two predetermined categories. The two predetermined categories include a by every 1% change in HbA1c value category and a by binary change in HbA1c value category. In some embodiments, the by every 1% change in HbA1c value category includes a 6% to 7% change bin, a 7% to 8% change bin, an 8% to 9% change bin, a 9% to 10% change bin, a 10% to 11% change bin, an 11% to 12% change bin, and a greater than or equal to 12% change bin. In some embodiments, the by binary change in HbA1c value category includes an adequate glycemic control bin and a poor glycemic control bin. In some embodiments, the adequate glycemic control bin includes an HbA1c value in the range of 6% to 9% and the poor glycemic control bin includes an HbA1 c value greater than or equal to 9%.

At step 106, the method 100 includes obtaining a plurality of total fracture incidences for the plurality of T2D subjects over a follow-up period. In some embodiments, the follow-up period is in the range of one year to three years. In some embodiments, the follow-up period is two years.

At step 108, the method 100 includes performing a plurality of linear regression model correlations between the binned HbA1c values and the plurality of total fracture incidences to determine a bone fracture rate prediction model. In some embodiments, the plurality of linear regression model correlations includes a Kaplan-Meier survival estimation model and a Cox proportional hazards model. In some embodiments, the Kaplan-Meier survival estimation model is a univariate model. In some embodiments, the Cox proportional hazards model is a multivariate model. In some embodiments, the multivariate model includes the variables of age, gender, comorbidities, glucocorticoids use, previous fragility fractures during the observational period, and prevalent T2D. In some embodiments, the bone fracture rate prediction model performs the univariate Kaplan-Meier survival estimation model to determine an initial bone fracture rate predictor, and then performs the multivariate Cox proportional hazards model to refine the initial bone fracture rate predictor for improved prediction accuracy, as discussed in more detail below.

At step 110, the method 100 includes obtaining a measurement of an HbA1c value of the T2D patient. At step 112, the method 100 includes determining the T2D patient's risk of bone fracture for a future period by analyzing the HbA1c value of the T2D patient with the bone fracture rate prediction model. In some embodiments, the future period is in the range of one year to three years. In some embodiments, the future period is two years.

In some embodiments, at step 114, the method 100 includes obtaining diabetic treatment information for the T2D patient, and at step 116, the method 100 includes adjusting the T2D patient's risk of bone fracture based on the diabetic treatment medication information. In some embodiments, the diabetic treatment medication information includes metformin and the T2D patient's risk of bone fracture is decreased by about 12% from an initial value. In some embodiments, the diabetic treatment medication information includes dipeptidyl peptidase-4 (“DDP4”) inhibitors and the T2D patient's risk of bone fracture is decreased by about 7% from an initial value. In some embodiments, the diabetic treatment medication information includes meglitinides and the T2D patient's risk of bone fracture is increased by about 12% from an initial value. In some embodiments, the diabetic treatment medication information includes thiazolidinediones (“TZDs”) and the T2D patient's risk of bone fracture is increased by about 20% to about 23% from an initial value. In some embodiments, the diabetic treatment medication information includes insulin and the T2D patient's risk of bone fracture is increased by about 24% to about 26% from an initial value. In some embodiments, the diabetic treatment medication information includes bisphosphonates and the T2D patient's risk of bone fracture is increased by about 15% from an initial value.

In some embodiments, the method uses de-identified administrative claims and electronic health records (“EHR”) data with linked laboratory results from the OptumLabs Data Warehouse. The database contains longitudinal health information on enrollees and patients, representing a diverse mixture of ages, ethnicities, and geographical regions across the United States. The data in OptumLabs Data Warehouse includes medical and pharmacy claims, laboratory results, and enrollment records for commercial and Medicare Advantage enrollees.

The EHR—derived data includes a subset of EHR data that has been normalized and standardized into a single database.

In some embodiments, a cohort study was conducted using de-identified data extracted for a period from Jan. 1, 2007, to Sep. 30, 2015. This study period was selected to ensure the EHR data were available for the period covered by the International Classification of Diseases, Ninth Revision (“ICD-9”) system. The index date for each individual was defined as the date of the first T2D diagnosis. The study cohort was further defined by using the following inclusion criteria: (1) age≥55 years with known gender; (2) at least 3-year continuous enrollment of Medicare Advantage/Commercial coverage with available medication information; (3) at least 2 inpatient or outpatient visits with T2D diagnosis within 1 year from the index date; and (4) at least 1 HbA1c measurement within the 2-year observational period starting at 15 days before the first T2D diagnosis. For each individual with multiple HbA1c records, the HbA1c values were averaged. Since the American Diabetes Association suggests a 6.5% HbA1c threshold for diabetes, people with a mean HbA1c value less than 6% were not considered in the study. Patients with only 1 HbA1c measurement were not excluded, as doing so might potentially bias the population against the mild/moderate T2D cases, thereby limiting the applications of these findings to this clinically relevant cohort. Also, since the first HbA1c measurement was recorded a few days before or on the day of the first diagnosis, this requirement ensures that the HbA1c value provides a reasonable approximation of glycemic control over the longer 2-year longitudinal period as opposed to a single value measured in close proximity or at the time of fracture. Eventually, a total of 157,439 individuals, selected from 4,018,250 people with T2D, were included in the study cohort, as shown in Table 1 below.

TABLE 1
Attrition table and cohort population

| | N |
|---|---|
| Individual with T2D diagnosis between 2007 and 2013 | 4,018,250 |
| Individual with at least 2 diagnosis service dates within 365 days | 3,027,830 |
| Individual meet age requirement and 3-year continuous enrollment | 557,627 |
| Individual with at least 1 HbA1c value within 730 days following first diagnosis | 187,185 |
| Individual with mean HbA1c value over 6% (inclusive) | 157,439 |

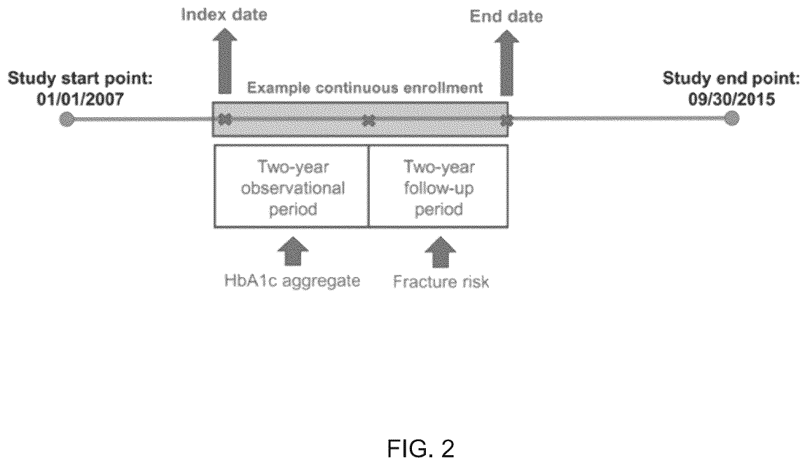

The study period is illustrated in FIG. 2. The mean HbA1c value for each individual in the cohort was binned into 2 types of predetermined categories: by every 1% change (6%-7%, 7%-8%, 8%-9%, 9%-10%, 10%-11%, 11%-12%, ≥12%), and by binary change (adequate glycemic control [6%-9%], poor glycemic control [≥9%]). In some embodiments, other classifications of adequate/poor glycemic control using 7% and 8% as cutoff were also examined. The standard deviation was calculated for people with more than 1 HbA1c record. If the individual had any HbA1c measurement during the follow-up period (third to fourth year), the values in follow-up period up to the first fracture incidence (if applicable) were also averaged and binned in the same manner.

To determine whether the duration of diabetes history may impact the association between fracture risk and longitudinal HbA1c, any diagnosis records of T2D for a period of 1 year prior to the study period (Jan. 1, 2006 to Dec. 31, 2006), were extracted and analyzed. If an individual had previous T2D diagnosis prior to the selected study period, the patient was considered to have longer history of T2D and was classified as with a case of prevalent T2D. Other individuals, who had the first T2D diagnosis during the study period, were classified as cases of incident T2D.

For a 2-year period from the index date, the records of antidiabetic and anti-osteoporotic medications were extracted. These included classes of biguanides (metformin), insulin, TZDs, sulfonylureas, meglitinides, DPP4 inhibitors, GLP-1 receptor agonists, α-blockers, bisphosphonates, selective estrogen receptor modulators (raloxifene), and estrogens. Other medications of interest, such as sodium-glucose cotransporter-2 inhibitors, teriparatide, and denosumab, were not included, as the drug classes were not approved by the Food and Drug Administration for use before the start of the study period (Jan. 1, 2007). The diagnosis records of diabetic comorbidities were also extracted for the entire study period. The selected comorbidities, known to directly affect fracture risk, included osteoporosis, neuropathy, retinopathy, nephropathy, coronary artery disease, hypertension, stroke, and obesity (body mass index [“BMI”]≤30). The use of glucocorticoids and a history of fragility fractures in the observational period were also considered as covariates. The total fracture incidences, as well as the total fragility fracture incidences, were obtained for a follow-up period of 2 years from the start of the third year to the end of fourth year after the index date. The fragility fracture incidences were defined based on the fracture site (with pathological fractures included) per ICD-9 codes, and these do not refer to the nature of the trauma. Individuals who did not sustain any fracture during the continuous enrollment period were censored.

In some embodiments, to further understand the relationship between bisphosphonates use and fracture risk, a nondiabetic control cohort was identified by randomly selecting 30% of commercial and Medicare Advantage enrollees for the same study period as the T2D group identified above. Individuals in the nondiabetic control group had no prior diagnosis of T2D, or prescription for insulin and/or thiazolidinediones. In this group, the use of bisphosphonates and fracture incidences were recorded and compared against the T2D cohort for identical periods.

Within the 2-year follow-up period, the Kaplan-Meier estimation of fracture probability was computed as a univariate model using two different predetermined categories of HbA1c bins (by every 1% change or by adequate [6%-9%] vs poor glycemic control [≥9%]). Log-rank tests were performed on HbA1c groups to verify if the difference in fracture risk was significant. The Kaplan-Meier model was also applied to three additional cases: (1) the risk estimation of fragility fractures, based on mean HbA1c; (2) the risk estimation for all fractures based on mean HbA1c during the follow-up period but before an individual's first fracture incidence (if applicable); and (3) the risk estimation of all fractures, stratified by higher or lower standard deviation of HbA1c from the cohort mean during the observational period.

Multivariate Cox proportional hazard models were employed to further investigate the instantaneous correlation between the fracture risk and the HbA1c groups, adjusted by various covariates. The hazard ratio “(h(t))” with a 95% CI is calculated, indicating the instantaneous risk of suffering an event, i.e., fracture, at any given time “t,” corresponding to each variable. In these models, the following variables were selected for adjustment to normalize their confounding effects on fracture risk: age, gender (male coded as 1 and female coded as 0), comorbidities (yes or no), glucocorticoids use (yes or no), and previous fragility fractures during the observational period (yes or no). Furthermore, an additional covariate— prevalent T2D (yes or no)—was included to examine whether a longer diabetes history would affect the relationship between HbA1c with fracture risk.

The association of medication use was similarly evaluated by including longitudinal HbA1c categories as an additional covariate. For antidiabetic treatments, the association was also analyzed after the exclusion of people with osteoporosis diagnosis. To examine the possibility of confounding by indication, the association of bisphosphonates use with fracture risk was also evaluated in a nondiabetic control cohort defined above after adjustments of age, gender, comorbidities (excluding osteoporosis), glucocorticoids use, and previous fracture. Due to the high collinearity in the Cox proportional hazards model of nondiabetic control group, a penalizer of 0.3 was added in both T2D and nondiabetic models. The hazard ratios of bisphosphonates use were compared between the T2D and nondiabetic individuals.

In some embodiments, to further understand the above relationships in different racial/ethnic groups, the fracture risk among Asian, Black, Hispanic, and White populations were compared. The comparisons were done with or without the adjustments of HbA1c categories and the confounding variables described above. Furthermore, Cox proportional hazards models were also applied to evaluate the association of HbA1c with fracture risk for the study population for each racial/ethnic group.

In some embodiments, data integration and organization were done using DbVisualizer software (DbVis Software AB, Stockholm, Sweden), and the statistical analyses were conducted in Python. A P value less than 0.05 was considered significant.

In an exemplary embodiment, 4,018,250 people with T2D were identified. Based on the inclusion criteria described above, a cohort of 157,439 individuals (50% male, 50% female) was identified for this study, as shown in Table 1 above. Table 2 below shows the demographics of the cohort. The mean age was 66.0 years with a standard deviation of 7.3 years. Among the 8,148 claims associated with a BMI value, 8.9% (N=726) of individuals had BMI under 25; 17.2% (N=1,402) of individuals had BMI between 25 and 29; and the remaining 73.9% (N=6,020) individuals passed the BMI threshold of 30. Using the HbA1c binning described above, more than half of the individuals were in the 6% to 7% HbA1c group (54.8%, N=86,211) and a quarter of the study individuals were in 7% to 8% range (25.5%, N=40,204). The 4 bins classifying people with HbA1c≥9% accounted to a total of 9.4% (N=14,839). In the study cohort, 79.9% (N=125,802) individuals were taking one or more classes of antidiabetic medication; and 10.4% (N=16,416) were taking one or more classes of anti-osteoporosis medication. About 27.2% (N=42,807) of the study population had T2D diagnosis prior to the study period. The numbers of patients with established comorbidities are also described in Table 2. For the first 2 years of the follow-up period, a total of 18,826 claims were extracted based on an individual's first fracture date. Among these claims, vertebrae fracture was most prevalent (13.1%, N=2,458, pathological fractures included), followed by hip fracture (9.8%, N=1,854, pathological fractures included). The prevalence of common fracture sites is listed in Table 3 below.

TABLE 2
Demographics table
Overall population N = 157,439

| Age | Mean | Std |
|---|---|---|
| | 66.04 | 7.27 |

| | | | Use of antidiabetic | | |
|---|---|---|---|---|---|
| Age categories | N | % | medications | N | % |
| 55-59 | 37,940 | 24.1% | All classes | 125,802 | 79.9% |
| 60-64 | 31,902 | 20.3% | Metformin | 95,342 | 60.6% |
| 65-69 | 35,062 | 22.3% | Insulin | 37,587 | 23.9% |
| 70-74 | 22,781 | 14.5% | TZD | 27,860 | 17.7% |
| 75-79 | 25,695 | 16.3% | Sulfonylureas | 59,542 | 37.8% |
| ≥80 | 4,059 | 2.6% | DPP-4 inhibitors | 22,504 | 14.3% |
| Sex | N | % | GLP-1 receptor agonists | 6,740 | 4.3% |
| Male | 78,530 | 49.9% | α-blockers | 882 | 0.6% |
| Female | 78,909 | 50.1% | Meglitinides | 2,870 | 1.8% |
| Race | N | % | Use of other medications | N | % |
| Asian | 8,195 | 5.2% | Biphosphonates | 9,122 | 5.8% |
| Black | 25,647 | 16.3% | Raloxifene | 1,431 | 0.9% |
| White | 99,056 | 62.9% | Estrogens | 6,797 | 4.3% |
| Hispanic | 14,888 | 9.5% | Glucocorticoids | 32,844 | 20.6% |
| Other/Unknown | 9,653 | 6.1% | Comorbidities | N | % |
| HbA1c bins | N | % | Neuropathy | 40,062 | 25.4% |
| 6%-7% | 86,211 | 54.8% | Retinopathy | 30,006 | 19.1% |
| 7%-8% | 40,204 | 25.5% | Nephropathy | 40,371 | 25.6% |
| 8%-9% | 16,185 | 10.3% | Coronary artery disease | 47,443 | 30.1% |
| 9%-10% | 7,525 | 4.8% | Stroke | 30,072 | 19.1% |
| 10%-11% | 3,752 | 2.4% | Hypertension | 146,113 | 92.8% |
| 11%-12% | 1,933 | 1.2% | Obesity | 35,442 | 22.5% |
| ≥12% | 1,629 | 1.0% | Osteoporosis | 16,756 | 10.6% |
| T2D history | N | % | Previous fragility fractures | N | % |
| | | | (observational period) | | |
| Prevalent T2D | 42,807 | 27.2% | | | |
| Incident T2D | 114,632 | 72.8% | Previous fractures | 4,965 | 3.2% |

TABLE 3
Common fracture sites for the 2-year follow-up period
All claims since individual first fracture date = 18,826

| Common fracture sites | N | % |
|---|---|---|
| Vertebrae | 2,458 | 13.1% |
| Femoral neck | 1,854 | 9.8% |
| Rib | 1,729 | 9.2% |
| Humerus | 1,696 | 9.0% |
| Radius or ulna | 1,584 | 8.4% |
| Ankle | 1,565 | 8.3% |
| Tarsal or metatarsal | 1,483 | 7.9% |
| Phalanges (foot) | 1,027 | 5.5% |
| Tibia or fibula | 829 | 4.4% |
| Carpal or metacarpal | 697 | 3.7% |
| Phalanges (hand) | 573 | 3.6% |
| Facial bones | 604 | 3.2% |
| Femoral shaft | 521 | 2.8% |
| Pelvis | 507 | 2.7% |

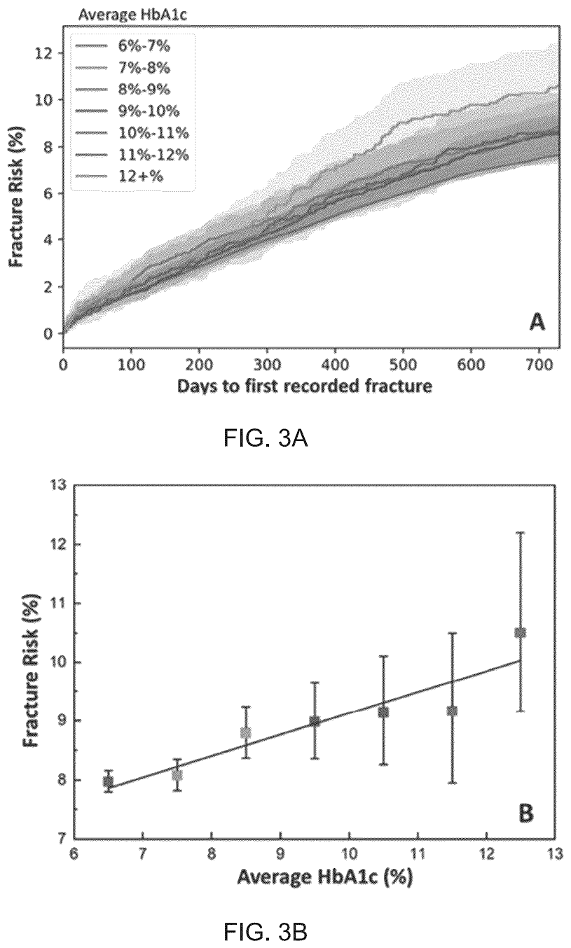

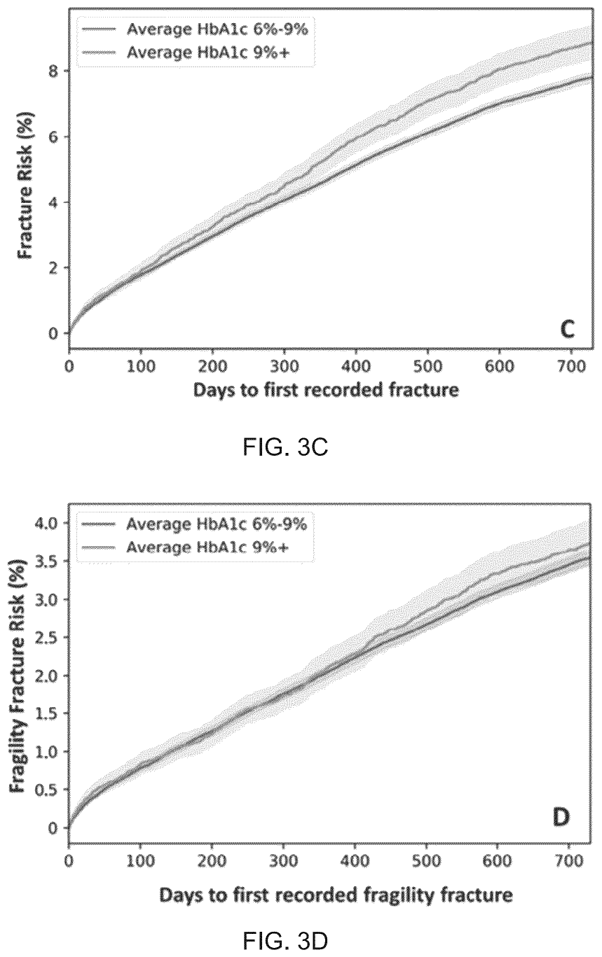

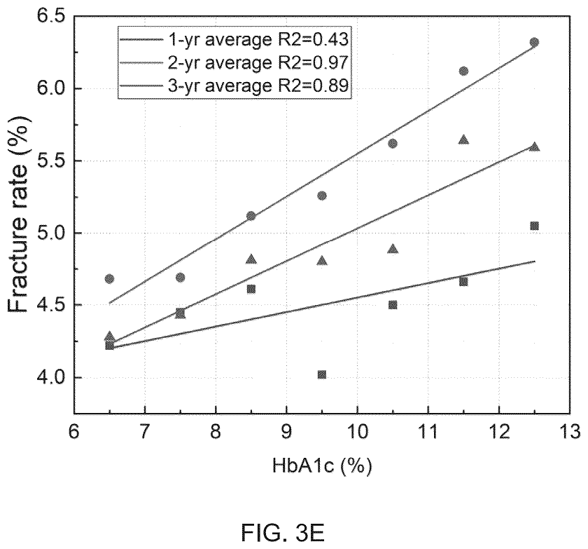

A univariate model was used to estimate the fracture probability in different longitudinal HbA1c cohorts. FIG. 3A demonstrates a Kaplan-Meier survival estimation of 2-year cumulative risk of all fractures with 95% CI (stratified by longitudinal HbA1c bins per 1% change) after the 2-year observation period. Multi-group log-rank test indicated that the differences in fracture risk were statistically significant between longitudinal HbA1c groups from the observational period (P value<0.001). At the end of the 2-year follow-up period, the cumulative fracture rate shows a significant increasing linear trend as HbA1c level increases (FIG. 3B, P=0.003, R=0.93). FIG. 3C shows the estimation of fracture risk when HbA1c bins were classified as an adequate glycemic control group and a poor glycemic control group. Here, the group with poor glycemic control had significantly higher risk of fracture than the group with adequate glycemic control (P<0.001). In contrast, the unadjusted longitudinal HbA1c did not have significant association with fragility fracture risk, as the fragility fracture risk was not statistically different between the adequate and poor glycemic control groups (P=0.23, FIG. 3D). FIG. 3E shows the follow-up fracture rate based on one-, two-, and three-years of HbA1c aggregation. The relationship between the one-year average HbA1c and fracture rate at follow-up is not significant, while the two- and three-year HbA1c relationships are significant. The two-year average HbA1c showed the strongest association; while the one- and three-year HbA1c underestimated the risk.

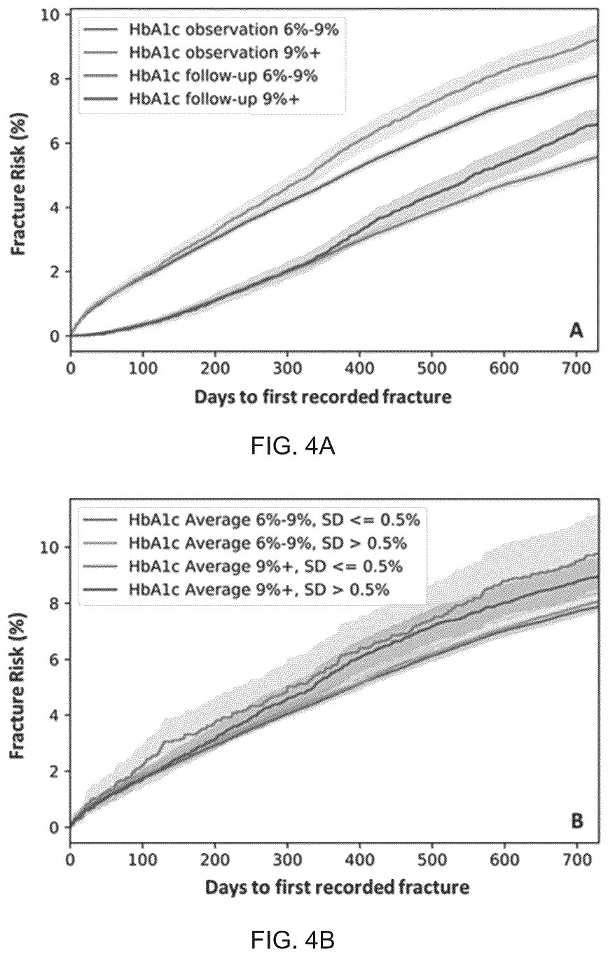

To show the value of utilizing two-year HbA1c instead of HbA1c measurement near fracture incidences, the mean of HbA1c for both the observational period and follow-up period was used to estimate risk of all fractures. As shown in FIG. 4A, the fracture risk, assessed by mean HbA1c from the follow-up period, was severely underestimated in comparison to the assessment using two-year longitudinal HbA1c from the observational period. Moreover, within the first year of the first fracture incidence during the follow-up period, the fracture risk cannot be stratified by glycemic control levels.

FIG. 4B shows the impact of variation in the HbA1c values during the 2-year observational period on the relationship between longitudinal glycemic control and fracture risk. Here, only individuals with more than one HbA1c measurement during the observational period were included in the analysis. Among this group, the mean SD for two-year HbA1c values was Therefore, we used a SD of 0.5% as a cutoff for separating the high and low variation of HbA1c groups. For both poor and adequate glycemic control groups, the difference in fracture risk was not statistically significant when stratified by SD.

To exclude the effects of covariates on HbA1c, a multivariate Cox proportional hazard model was applied. Table 4 below shows the hazard ratio of longitudinal HbA1c stratified by every 1% change and by adequate (6%-9%) vs poor (>9%) glycemic control. The adjustment was done minimally (adjusted for age, gender, glucocorticoids use, and previous fracture), and with multiple covariates (adjusted for age, gender, glucocorticoids use, previous fracture, osteoporosis, neuropathy, retinopathy, nephropathy, coronary artery disease, hypertension, stroke, and obesity). The hazard ratio was 1.08 for each 1% increase in longitudinal HbA1c, (95% CI [1.07, 1.10]; P<0.001). This indicates that for each 1% increase in two-year HbA1c there was concomitant 8% increase in fracture risk for the following two years. When further adjusted for various comorbidities, the hazard ratio attenuated to 1.05 (95% CI [1.03, 1.06]; P<0.001). Compared with the group with adequate glycemic control, the group with poor glycemic control had a 29% increase in fracture risk when adjusted minimally (hazard ratio: 1.29; 95% CI [1.22, 1.36]; P<0.001), and a 19% increase in fracture risk when further adjusted for additional comorbidities (hazard ratio: 1.18; 95% CI [1.11, 1.25]; P<0.001). Adjusting the cutoff for poor glycemic control to >7% and >8% resulted in a change of the minimally adjusted hazard ratio to 1.13 (CI [1.09, 1.17]; P<0.001) and 1.20 (CI [1.15, 1.25]; P<0.001), respectively. Separating fractures as incident or prevalent (or by excluding prevalent T2D cohort) did not alter the hazard ratio determined using longitudinal HbA1c.

TABLE 4
Hazard ratio of longitudinal HbA1c in two binning methods

| | Minimally adjusted hazard | Multivariate adjusted hazard |
|---|---|---|
| Longitudinal HbA1c | ratio (95% CI) | ratio (95% CI) |
| Per 1% increase of | 1.08 (1.07, 1.10) | 1.05 (1.03, 1.06) |
| longitudinal HbA1c | | |
| From adequate to poor | 1.29 (1.22, 1.36) | 1.18 (1.11, 1.25) |
| glycemic control | | |

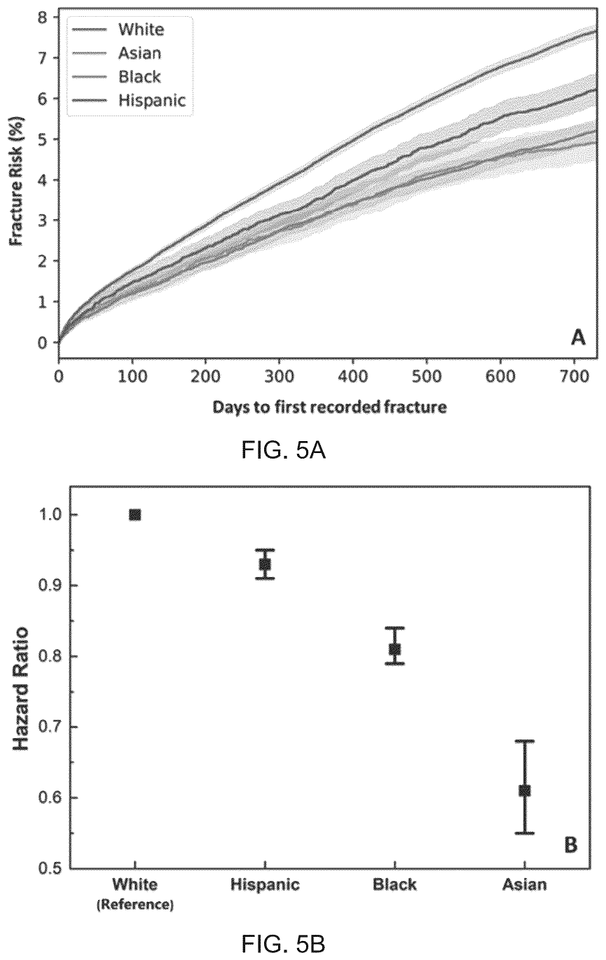

FIG. 5A demonstrates the unadjusted fracture risk assessment in the White, Asian, Black, and Hispanic populations. The fracture risk was significantly higher in the White population than the other three racial groups. The Black population had higher risk of fracture than the Asian and Hispanic populations, while the risk was not significantly different between the Asian and Hispanic groups. With confounding variables adjusted, compared with the White population (hazard ratio set to 1, as shown in FIG. 5B), the fracture risk was lowered by 7% in the Hispanic population (hazard ratio: 0.93; CI [0.91, 0.95]), 19% in the Black population (hazard ratio: 0.81; CI [0.79, 0.84]), and 39% in the Asian population (hazard ratio: 0.61; CI [0.55, 0.68]). Interestingly, when comparing the effect of poor and adequate glycemic control on fracture risk in the four racial/ethnic groups separately, the relationship remained unchanged in the White and Black populations but was no longer significant in the Asian population with minimal adjustment, or in the Asian and Hispanic populations with multivariate adjustment, as shown in Table 5 below.

TABLE 5
Relationship between longitudinal HbA1c (poor/adequate glycemic control) and fracture
risk in four racial groups, minimally adjusted and multivariate adjusted

| From adequate to poor | Minimally adjusted hazard | Multivariate adjusted hazard |
|---|---|---|
| glycemic control | ratio (95% CI; P value) | ratio (95% CI; P value) |
| Asian | 1.33 (0.95, 1.87; 0.09) | 1.20 (0.84, 1.70; 0.31) |
| Black | 1.25 (1.08, 1.44; 0.002) | 1.19 (1.02, 1.38; 0.02) |
| Hispanic | 1.21 (1.01, 1.45; 0.04) | 1.10 (0.92, 1.33; 0.30) |
| White | 1.32 (1.22, 1.42; <0.001) | 1.21 (1.13, 1.31; <0.001) |

The significant association with medications use, adjusted for multiple variables listed above, are shown in Table 6 below. The use of metformin and DDP4 inhibitors were associated with a 12% decrease (hazard ratio: 0.88; 95% CI [0.85, 0.92]; P=0.003), and a 7% decrease (hazard ratio: 0.93; 95% CI [0.88, 0.98]; P=0.005) in fracture risk, respectively. In contrast, meglitinides, TZDs, and insulin use were correlated with 12%, 20%, and 24% higher fracture risk, respectively (hazard ratio for meglitinides: 1.12; 95% CI [1.00, 1.26]; P=0.04; hazard ratio for TZDs: 1.23; 95% CI [1.18, 1.29]; P<0.001; hazard ratio for insulin: 1.26; 95% CI [1.21, 1.32]; P<0.001). Interestingly, bisphosphonates use was also associated with a 15% increase in fracture risk within the T2D group (hazard ratio: 1.15; 95% CI [1.07, 1.22]; P<0.001). However, the elevated fracture risk in the T2D group was not different than in the nondiabetic group, as the difference between hazard ratios was not statistically significant (hazard ratio in T2D group: 1.16; 95% CI [1.13, 1.20]; hazard ratio in nondiabetic control group: 1.13; 95% CI [1.11, 1.14], both P<0.001). Exclusion of the osteoporotic cohort from the analysis (instead of adjusting for osteoporosis condition) did not significantly alter the hazard ratios for the antidiabetic treatments. Other antidiabetic medications (sulfonylureas, GLP-1 receptor agonists, a-blockers) and other anti-osteoporotic medications (raloxifene and estrogens) did not show any significant impact on fracture risk.

TABLE 6
Hazard ratios of medications use

| | | | | Hazard | |
|---|---|---|---|---|---|
| | Medication | Medication | Associated | ratio (95% | P- |
| | class | generics | with | CI) | value |
| Anti- | Biguanides | Metformin | Decreased | 0.88 (0.85, | 0.003 |
| diabetic | | | risk | 0.92) | |
| | DPP Inhibitors | Sitagliptin, | Decreased | 0.93 (0.88, | 0.005 |
| | | linagliptin, | risk | 0.98) | |
| | | saxagliptin, | | | |
| | | alogliptin | | | |
| | Insulin | Insulin | Increased | 1.26 (1.21, | <0.001 |
| | | | risk | 1.32) | |
| | TZDs | Pioglitazone, | Increased | 1.23 (1.18, | <0.001 |
| | | rosiglitazone | risk | 1.29) | |
| | Meglitinides | Nateglinide, | Increased | 1.12 (1.00, | 0.04 |
| | | repaglinide | risk | 1.26) | |
| | GLP-1 Receptor | Exenatide, | No | 1.06 (0.97, | 0.20 |
| | Analogues | liraglutide, | association | 1.15) | |
| | | lixisenatide, | | | |
| | | dulaglutide, | | | |
| | | semaglutide | | | |
| | α-Glucosidase | Acarbose, | No | 0.94 (0.75, | 0.55 |
| | Inhibitors | miglitol | association | 1.17) | |
| | Sulfonylureas | Glimepiride, | No | 0.99 (0.95, | 0.47 |
| | | glipizide, | association | 1.02) | |
| | | tolazamide, | | | |
| | | tolbutamide, | | | |
| | | glyburide, | | | |
| | | chlorpropamide | | | |
| Anti- | Bisphosphonates | Alendronate, | Increased | 1.15 (1.07, | 0.004 |
| resorptive | | ibandronate, | risk | 1.22) | |
| | | risedronate, | | | |
| | | zoledronic acid | | | |
| | Selective | Raloxifene | No | 0.91 (0.77, | 0.23 |
| | estrogen | | association | 1.07) | |
| | receptor | | | | |
| | modulators | | | | |
| | Estrogens | Estradiol, | No | 1.06 (0.98, | 0.16 |
| | | conjugated | association | 1.14) | |
| | | estrogens, | | | |
| | | estropipate | | | |

The increased fracture risk in people with T2D is not accurately evaluated by a BMD-based assessment, and the efficacy of common medications in rescuing type 2 diabetic fracture risk is not established. The present technology demonstrates a significant independent correlation between longitudinal HbA1c, commonly used medications, and two-year fracture risk in a large cohort with 157,439 T2D individuals.

The present technology has at least several strengths. First, it uses a nationwide database containing more than 4 million commercial and Medicare Advantage enrollees with T2D, with a broad distribution of geographical regions and races. Second, all clinical fractures were evaluated, including typical fracture types attributed to T2D, such as hip and vertebrae fractures. Third, embodiments identified a universal, and cost-effective blood-based measurement, HbA1c, as an effective predictor for fracture. Accordingly, the present technology demonstrates the superiority of using two-year longitudinal HbA1c over HbA1c measured close to fracture incidences.

The association between the longitudinal HbA1c and the fracture risk is more evident and more physiologically relevant than fasting blood glucose or single time point HbA1c measurements either at baseline or just prior to fracture. For example, one of the essential mechanisms for the association between long-term poor glycemic control and fracture could be nonenzymatic glycation (“NEG”). NEG is a systemic diffusion-based process where the accumulation of advanced glycation end-products (“AGEs”) is driven by blood glucose concentration and the time of exposure seen commonly with poor glycemic control in T2D. Accumulation of AGEs through NEG disrupts bone turnover and alters bone quality. Mechanistically, the two-year longitudinal HbA1c may serve as a suitable indicator of the level of deterioration in bone tissue from elevated blood glucose over time, and may therefore associate with fracture risk, as is shown herein.

In some embodiments, over the two-year period, also included were the individuals with only 1 HbA1c measurement (N=42,783, 27.2%), since excluding this subgroup might bias the population against the mild/moderate T2D cases. By excluding this subgroup, the change to this association between HbA1c and fracture risk is minimal (hazard ratio for poor to adequate glycemic control, minimally adjusted: 1.28; 95% CI [1.16, 1.41], vs 1.29; 95% CI [1.22, 1.36] without exclusion). This result suggests that the association between HbA1c and fracture risk is valid as long as there is at least 1 HbA1c record extended across two years, as opposed to a HbA1c value measured near to fracture.

The univariate model distinguished the time-to-fracture probability in people with different values of longitudinal glycemic levels and provided a method to assess fracture risk solely based on a relatively short term (two-year) HbA1c level. In contrast, two-year longitudinal HbA1c did not significantly stratify risk of fragility fractures (defined based on fracture sites only, not referring to the degree of trauma). Such an outcome can be partially explained due to the reduced size of cohort with fractures at specific sites associated with fragility (N=13,309 by the end of the 2-year follow-up, compared with N=58,510 for all fractures). More importantly, selecting only fragility fracture sites could increase the proportion of fractures caused by osteoporosis within the cohort, and, unlike T2D fractures, the occurrence of osteoporotic fractures may be dominated by factors other than glycemic control. The present technology shows that the association with longitudinal HbA1c and fracture risk is significant when all fractures in the T2D population are considered.

In some embodiments, once the HbA1c mean is controlled, the variation and fluctuation of HbA1c within the two-year period do not have a statistical impact on fracture risk. This outcome may be explained by noting that the impact of glycemic control on bone via a diffusion-based process, such as NEG, is dominated by average value over a period. Consequently, once the mean glycemic level is reached over a moderate period, the fluctuation of HbA1c may not have major effects on bone quality and bone turnover. Thus, our results suggest that people with high variance of HbA1c do not present higher risk of T2D fractures.

Furthermore, the multivariate model used in some embodiments of the present technology demonstrates the independent fracture risk of differences in longitudinal HbA1c. Some embodiments show that a 1% increase of longitudinal HbA1c is responsible for an 8% higher risk of fractures. After adjusting for multiple diabetic comorbidities, there is still a 5% increased fracture risk directly associated with a 1% elevation in longitudinal HbA1c. It is noteworthy that the seemingly low hazard ratio corresponds to a small difference in HbA1c (1%) within a relatively short two-year follow-up period. If the patient remains within a poorly controlled glycemic level for two years, the risk of fracture is increased by 29%, in comparison with patients maintaining adequate glycemic control. A 29% increase in fracture risk in the United States alone will account for more than 1 million additional fractures in people with poor glycemic control within a period of two years. Poor glycemic control can also account for the higher risk of fracture related to diabetic comorbidities. For example, the risk of developing retinopathy and/or neuropathy is dependent on the longitudinal HbA1c level. Both conditions could lead to higher risk of fall and hence increased fracture risk, although fracture risk in T2D remains unchanged after adjustment for higher fall incidences. For example, the present technology shows that two-year longitudinal HbA1c significantly correlated to the occurrence rate of other diabetic comorbidities (retinopathy R2=0.73, neuropathy R2=0.79, nephropathy R2=0.89, all P<0.01) and can therefore partially account for the increased fracture risk caused by diabetic comorbidities.

Table 5 above presents the evidence of the same relationship between poor/adequate glycemic control and fracture risk in different racial groups. With confounding factors fully adjusted, the hazard ratios between the Asian, Black, and White populations are indeed similar. The lack of statistical significance in both models for the Asian population could be attributed to the much smaller sample size (N=8,195). However, this relationship does seem to attenuate in Hispanic population. As the T2D fracture risk in different racial groups is essentially different (FIG. 5A), other factors, not included in the current model, such as bone structure and geometry, dietary habit, and socioeconomic status, are potential contributors to increased fracture risk in T2D.

In clinical practice, it is usually difficult to rigorously assess the patient's diabetes history, as precise information on the onset of T2D is generally not available. Here, some embodiments separated the cohort as an incident T2D group and a prevalent T2D group whose T2D history is potentially longer. The prevalent T2D group had slightly higher unadjusted risk of fractures than the incident T2D group, possibly due to higher mean age. However, exclusion of prevalent T2D cohort or its inclusion as a covariate did not alter the relationship between longitudinal HbA1c and fracture risk in the multivariate model. Two previous studies show that bone material strength and fracture risk are impacted by the duration of diabetes. However, these studies did not adjust for long-term glycemic levels and, when adjusted for long-term average HbA1c, the duration of diabetes was no longer found to be associated with bone material strength. The present technology shows that the two-year HbA1c observational window is indeed sufficient to account for any impact of diabetes duration and to adequately evaluate the fracture risk. These findings emphasize the clinical importance of proper maintenance of glycemic control in reduction of fracture risk with T2D.

In terms of medications, six types of medications selected significantly correlated with the alteration of fracture risk after adjusting for multiple covariates, as shown in Table 6 above. Metformin was associated with a 12% lower fracture risk. It is noteworthy that metformin not only lowers HbA1c level, but it also reverses the negative effects of AGEs on osteoblastic cells and normalizes the bone forming process. Similarly, in preclinical studies, GLP-1 receptor agonists stimulate osteoblast differentiation while suppressing osteoclast activities. However, the present technology did not observe any significant alteration in fracture risk associated with GLP-1 receptor agonist use. DPP4 inhibitors were associated with a 7% decrease in fracture risk. Conversely, use of TZDs damages osteoblasts and subsequently leads to decrease in BMD, and thus the present technology found that the use of TZDs was indeed correlated to a 23% increase in all fracture risk.

Insulin use was associated with a 26% increase in fracture risk. In particular, peripheral hyperinsulinemia, induced by insulin use in T2D, negatively influences osteoclastogenesis. The resulting impairment in bone turnover from hyperinsulinemia could therefore result in higher fracture risk. Insulin users may also have higher level of severity in diabetes/diabetic complications that are not accounted for covariates considered herein. Although the difference in longitudinal HbA1c was normalized when considering the effects of medications, insulin use can also lead to temporary hypoglycemia (subsequently recovered) and a consequent increase in the risk of fall related fractures. Interestingly, sulfonylureas, which is also likely to induce temporary hypoglycemia and known to increase fracture risk, appeared to have no association with fracture risk in some embodiments of the present technology. There is currently no clinical evidence on fracture risk with 2 classes of antidiabetic medication use (meglitinides and α-blockers). The present technology revealed that meglitinides are associated with a 12% increase in all fracture risk. Thus, careful evaluations of bone fractures in relation to meglitinides is suggested for future studies.

Anti-osteoporotic medications are primarily used to prevent fragility fractures in osteoporosis by normalizing bone turnover. While raloxifene and estrogens did not impact T2D associated fracture risk, the present technology demonstrated a 15% increase in fracture risk with bisphosphonates use. Because the increased fracture risk associated with bisphosphonates use in T2D was similar to that in nondiabetic individuals, such association is likely due to confounding by indication. However, it is worth noting that, as bisphosphonates prevent fractures by inhibiting osteoclast activity, the increased fracture risk in bisphosphonates users in both T2D and control may be linked to suppression of bone turnover and attenuated remodeling with long-time use of bisphosphonates. Among the bisphosphonates users herein, prior to the follow-up period, 56% had cumulated use of bisphosphonates for more than a year while 31% had bisphosphonates therapy for more than two years. Therefore, prescribing bisphosphonates to people with poor glycemic control should be considered cautiously, on a case-by-case basis.

In some embodiments, an additional analysis was conducted where fractures, not directly attributed to T2D including skull fractures, finger fractures, toe fractures, and fractures at unspecified sites were excluded from the analyses presented here. The relationship between longitudinal HbA1c and fracture risk remained the same as discussed above, suggesting that the increased fracture risk in T2D found here is valid for multiple fracture sites associated with both high and low energy trauma.

Accordingly, the present technology indicates that longitudinal HbAl c measurement is a significant and effective tool for fracture risk assessment. The present technology determined that medication (metformin, insulin, TZDs, DPP4 inhibitors, meglitinides, and bisphosphonates) use in T2D population over a period of 2 years is associated with alteration in fracture risk for the following period. The present technology provides important clinical input on management and reduction of fracture risk in people with T2D through monitoring and management of the longitudinal glycemic control, and the use of metformin and/or DPP4 inhibitors.

As will be apparent to those skilled in the art, various modifications, adaptations, and variations of the foregoing specific disclosure can be made without departing from the scope of the technology claimed herein. The various features and elements of the technology described herein may be combined in a manner different than the specific examples described or claimed herein without departing from the scope of the technology. In other words, any element or feature may be combined with any other element or feature in different embodiments, unless there is an obvious or inherent incompatibility between the two, or it is specifically excluded.

References in the specification to “one embodiment,” “an embodiment,” etc., indicate that the embodiment described may include a particular aspect, feature, structure, or characteristic, but not every embodiment necessarily includes that aspect, feature, structure, or characteristic. Moreover, such phrases may, but do not necessarily, refer to the same embodiment referred to in other portions of the specification. Further, when a particular aspect, feature, structure, or characteristic is described in connection with an embodiment, it is within the knowledge of one skilled in the art to affect or connect such aspect, feature, structure, or characteristic with other embodiments, whether or not explicitly described.

The singular forms “a,” “an,” and “the” include plural reference unless the context clearly dictates otherwise. Thus, for example, a reference to “a plant” includes a plurality of such plants. It is further noted that the claims may be drafted to exclude any optional element. As such, this statement is intended to serve as antecedent basis for the use of exclusive terminology, such as “solely,” “only,” and the like, in connection with the recitation of claim elements or use of a “negative” limitation. The terms “preferably,” “preferred,” “prefer,” “optionally,” “may,” and similar terms are used to indicate that an item, condition, or step being referred to is an optional (not required) feature of the technology.

The term “and/or” means any one of the items, any combination of the items, or all of the items with which this term is associated. The phrase “one or more” is readily understood by one of skill in the art, particularly when read in context of its usage.

Each numerical or measured value in this specification is modified by the term “about.” The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values and ranges proximate to the recited range that are equivalent in terms of the functionality of the composition, or the embodiment.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percents of carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third, and upper third, etc.

As will also be understood by one skilled in the art, all language such as “up to,” “at least,” “greater than,” “less than,” “more than,” “or more,” and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the technology encompasses not only the entire group listed as a whole, but each member of the group individually and all possible subgroups of the main group. Additionally, for all purposes, the technology encompasses not only the main group, but also the main group absent one or more of the group members. The technology therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, as used in an explicit negative limitation.

What is claimed is:

1. A method of predicting bone fracture risk in a type 2 diabetes (“T2D”) patient, comprising:
obtaining a plurality of averaged glycated hemoglobin (“HbA1c”) values for a plurality of T2D subjects over an observational period;
binning the plurality of averaged HbA1c values into two predetermined categories;
obtaining a plurality of total fracture incidences for the plurality of T2D subjects over a follow-up period;
performing a plurality of linear regression model correlations between the binned HbA1c values and the plurality of total fracture incidences to determine a bone fracture rate prediction model;
obtaining a measurement of an HbA1c value of the T2D patient; and
analyzing the HbA1c value of the T2D patient with the bone fracture rate prediction model to determine the T2D patient's risk of bone fracture for a future period.

2. The method of claim 1, wherein the two predetermined categories comprise a by every 1% change in HbA1c value category and a by binary change in HbA1c value category.

3. The method of claim 2, wherein the by every 1% change in HbA1c value category comprises a 6% to 7% change bin, a 7% to 8% change bin, an 8% to 9% change bin, a 9% to 10% change bin, a 10% to 11% change bin, an 11% to 12% change bin, and a greater than or equal to 12% change bin.

4. The method of claim 2, wherein the by binary change in HbA1c value category comprises an adequate glycemic control bin and a poor glycemic control bin.

5. The method of claim 4, wherein the adequate glycemic control bin comprises an HbA1c value in the range of 6% to 9% and the poor glycemic control bin comprises an HbA1c value greater than or equal to 9%.

6. The method of claim 1, wherein the plurality of linear regression model correlations comprises a Kaplan-Meier survival estimation model and a Cox proportional hazards model.

7. The method of claim 6, wherein the Kaplan-Meier survival estimation model is a univariate model.

8. The method of claim 6, wherein the Cox proportional hazards model is a multivariate model.

9. The method of claim 8, wherein the multivariate model comprises the variables of age, gender, comorbidities, glucocorticoids use, previous fragility fractures during the observational period, and prevalent T2D.

10. The method of claim 1, wherein the observational period is in the range of one year to three years and the follow-up period is in the range of one year to three years.

11. The method of claim 1, wherein the observational period is two years and the follow-up period is two years.

12. The method of claim 1, wherein the future period is in the range of one year to three years.

13. The method of claim 1, wherein the future period is two years.

14. The method of claim 1, further comprising:
obtaining diabetic treatment medication information for the T2D patient; and
adjusting the T2D patient's risk of bone fracture based on the diabetic treatment medication information.

15. The method of claim 14, wherein the diabetic treatment medication information comprises metformin and the T2D patient's risk of bone fracture is decreased by about 12% from an initial value.

16. The method of claim 14, wherein the diabetic treatment medication information comprises dipeptidyl peptidase-4 (“DDP4”) inhibitors and the T2D patient's risk of bone fracture is decreased by about 7% from an initial value.

17. The method of claim 14, wherein the diabetic treatment medication information comprises meglitinides and the T2D patient's risk of bone fracture is increased by about 12% from an initial value.

18. The method of claim 14, wherein the diabetic treatment medication information comprises thiazolidinediones (“TZDs”) and the T2D patient's risk of bone fracture is increased by about 20% to about 23% from an initial value.

19. The method of claim 14, wherein the diabetic treatment medication information comprises insulin and the T2D patient's risk of bone fracture is increased by about 24% to about 26% from an initial value.

20. The method of claim 14, wherein the diabetic treatment medication information comprises bisphosphonates and the T2D patient's risk of bone fracture is increased by about 15% from an initial value.
